(19) United States
(12) Patent Application Publication
(10) Pub. No.: US 2009/0053769 A1
(43) Pub. Date: Feb. 26, 2009

(54) HETEROLOGOUS PRODUCTION OF NATURAL PRODUCTS IN BACTERIA

(75) Inventors: Clay C. C. Wang, Los Angeles, CA (US); Kenji Watanabe, Los Angeles, CA (US); Alex P. Praseuth, Los Angeles, CA (US)

Correspondence Address: HOGAN & HARTSON L.L.P., 1999 AVENUE OF THE STARS, SUITE 1400, LOS ANGELES, CA, 90067, US

(73) Assignee: University of Southern California, Los Angeles, CA (US)

(21) Appl. No.: 12/140,185

(22) Filed: Jun. 16, 2008

Related U.S. Application Data
(60) Provisional application No. 60/944,620, filed on Jun. 18, 2007.

(51) Int. Cl. C12P 21/00; C12N 1/21

(52) U.S. Cl. 435/71.3; 435/252.33

(57) ABSTRACT

The present invention demonstrates an example of the de novo total biosynthesis of biologically active forms of heterologous NRPs in Escherichia coli (E. coli). The system can serve not only as an effective and flexible platform for large-scale preparation of natural products from simple carbon and nitrogen sources, but also as a general tool for detailed characterizations and rapid engineering of biosynthetic pathways for microbial syntheses of novel compounds and their analogs.

The present application claims the benefit of the filing date of U.S. Provisional Application No. 60/944,620, filed Jun. 18, 2007, the disclosure of which is incorporated herein by reference in its entirety.

### FUNDING

This invention was made with support by grants from NIH GM 075857-01, American Cancer Society grant RSG-06-010-01-CDD, University wide AIDS Research Program ID05-USC-055, and Grant-in-Aids for Scientific Research from the Japan Society for the Promotion of Science (A) 17208010.

### FIELD OF THE INVENTION

The present invention relates to an E. coli-based total biosynthesis of a bioactive form of heterologous complex Nonribosomal Peptides (NRPs) from simple carbon and nitrogen sources.

### BACKGROUND OF THE INVENTION

Nonribosomal peptides (NRPs) encompassing vancomycin, cyclosporine A, echinomycin, and triostin A are celebrated components of a variety of microbial secondary metabolites possessing biological activities such as antibiotics, immunosuppressants, and antitumor agents immensely important for clinical use (1-4). This category's broad spectrum of natural products and its structural complexity are a result of them being biosynthesized by NRP synthetases (NRPSs) encoded in a single modulated megaenzyme ranging in size from 120 to 180 kDa. Each NRPS consists of three essential functioning domains: condensation, adenylation, and thiolation. These domains boast the ability to catalyze an amide bond formation using amino acids as building units for their peptide architecture (5-7). A single module of this megasynthetase may also carry a methylation domain for N-methylation of the peptide backbone and/or epimerization domain for switching an amino acid's stereochemistry during the peptide elongation process. The structural complexity and diversity of NRPs are largely attributed to these modules and methods in which they are synthesized.

Nature has provided a generous assortment of NRPs. However, the artillery of natural products as potential drugs or seeds of promising medication have their limitations due to insufficient material for investigative clinical trial. This obstacle can be attributed to the original host's low productivity or unavoidably expensive cost of multistep chemical syntheses to avoid undesirable byproducts and impart a favorable pharmacokinetic profile. During the past decade, many notable NRPs have been isolated from streptomycetes. Stimulated interests surrounding their mode of production compelled researchers to identify and sequence genes that are likely involved in this process. To understand the biosynthetic mechanisms, with aims for increasing the yield of production and intention of pursuing desired analogues, two agreeable expression systems using Streptomyces lividans and S. coelicolor as hosts with amplifiable expression vectors were developed by Hopwood et al. (8). These two systems have presented substantial results to provide lucid biological understanding of streptomycetes' proteins and the production of secondary metabolites, more markedly, polyketides (PKs), a category of natural products similar to NRPs in terms of how they are biosynthesized by modular macroenzymes (5). Although the contributions by the model hosts are influential to this field of research, construction of plasmid vectors and the cultivation of these cells are both time-consuming and problematic.

In recent years, progress has been made using E. coli as a surrogate host for gene expression of NRPSs and PK synthases (PKSs) (9). Positive results for heterologous production of anticipated compounds in E. coli were observed despite the use of Khosla's innovative but elaborate multiple-plasmid expression system (10-12). There are three great advantages of using E. coli as a heterologous host: (i) availability of a wealth of well-established molecular biological techniques for its genetic and metabolic manipulation, (ii) robust tolerance toward exogenous proteins and fast life cycle, and (iii) large-scale protein production ability, which will facilitate investigations for detailed reaction mechanisms of the biosynthetic pathway. Together, these advantages set the stage for engineering biosynthesis of desired compounds and its useful analogues through modification or mutation of the biosynthetic genes in a much shorter time frame.

The inventors recently established a de novo system by which echinomycin (1) and triostin A (2) (FIG. 1) were biosynthesized in a heterologous host, E. coli (13). Based on structural similarities of 1 and 2, and predictions centered around isolated sequenced genetic material extracted from the echinomycin producing bacterium, S. lasaliensis, compound 2 may possibly be the precursor of compound 1 (Scheme 1C). The inventors reported the enzymatic conversion of compound 2 to 1 in the presence of Ecm 18, an enzyme that is highly homologous to celebrated S-adenosyl-L-methionine dependent methyltransferases. Quinomycin antibiotics such as 1 and 2 are commonly distinguished by their bicyclic quinoxaline or quinoline chromophore attached to the C₂ symmetric peptide backbone and can be further discerned by the thioacetal cross-bridge on compound 1. These bicyclic chromophores or speculative starting units for biosyntheses of this class of natural products bestow them with their notable pharmaceutical bioactivity or ability to bisintercalate to DNA. This preceding report has presented proficient levels of these two NRPs, demonstrating the first successful production of complex NRPs employing intact biosynthetic pathways from S. lasaliensis in E. coli using only simple carbon and nitrogen sources. Moreover, to avoid impairing the host from the metabolite's toxicity or DNA-bisintercalating properties (14, 15), the inventors incorporated into our plasmid a hypothetical resistance-gene found in the biosynthetic gene cluster that is highly homologous to known daunorubicin resistance-gene (DrrC) into E. coli (16, 17). This resistance allowed the heterologous host to tolerate increased production of these antibiotics and allowed the isolation of these two compounds from the culture of recombinant E. coli. The model system also provided for the unraveling of additional details so that E. coli could be metabolically engineered for a more substantial production of biologically active NRPs and NRP analogues.

The inventors estimated the production of compound 2 using small-scale cultivation and a liquid chromatography-mass spectrometer (LC-MS) for quantitative analysis of the product under select culture conditions. They also compared the de novo production while furnishing QXC to corroborate this chromophore's role as the priming unit for biosynthesis of the quinomycin antibiotics (18, 19). This simple and speedy approach provided for valuable information for maximizing the production titer.

### SUMMARY OF THE INVENTION

The present invention provides examples of the de novo total biosynthesis of biologically active forms of heterologous NRPs in Escherichia coli (E. coli). The system can serve not only as an effective and flexible platform for large-scale preparation of natural products from simple carbon and nitrogen sources, but also as a general tool for detailed characterizations and rapid engineering of biosynthetic pathways for microbial syntheses of novel compounds and their analogs.

Proficient production of the antitumor agent triostin A was developed using engineered Escherichia coli (E. coli). The bacterium played host to 15 genes that encode integral biosynthetic proteins which were identified and cloned from Streptomyces lasaliensis. Triostin A production was dramatically increased by more than 20-fold, 13 mg/L, with the introduction of exogenous quinoxaline-2-carboxylic acid (QXC), the speculative starting unit for biosynthesis of triostin A. Conversely, de novo production of triostin A by means of high cell density fed-batch fermentation that is exclusive of exogenous QXC bore a modest amount of the antitumor agent. Noteworthy production of the biologically active molecule was achieved with small-scale cultivation and quantitative analysis of the product was accomplished with a liquid chromatography-mass spectrometer. This simple and speedy approach provided for valuable information for maximizing the production titer. The entirely heterologous production system also establishes a basis for the future use of E. coli for generation of novel bioactive compounds through tolerable precursor-directed biosynthesis.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE FIGURES

FIG. 1. Chemical structures of quinomycin antibiotics.

FIG. 2. The echinomycin biosynthetic cluster from Streptomyces lasaliensis. (a) Echinomycin biosynthetic gene cluster from Streptomyces lasaliensis. (b) Predicted fatty acid synthase gene organization in S. lasaliensis. (c) Deduced functions of the ORFs of the S. lasaliensis echinomycin biosynthetic gene cluster and fatty acid synthase acyl carrier protein. Expect value is the number of matches expected to be found purely by chance for a given query sequence in a database (20). n.a.: not applicable; QC: quinoxaline-2-carboxylic acid.

FIG. 3. HPLC-MS analyses of the Ecm 18-catalyzed thioacetal formation. (a) Mechanism for the formation of thioacetal bridges with alkyl substituents of differing lengths. (b) HPLC-MS analysis of the crude extract of the reaction mixture for the Ecm 18-catalyzed conversion of 2 to 1. UV (λ=254 nm) trace is shown in inset. (c) HPLC-MS analysis of 1 isolated from the crude extract of the reaction mixture. (d) HPLC-MS analysis of 2 isolated from the crude extract of the reaction mixture.

FIG. 4. Chemical characteristic spectra of compound 1 produced by the engineered E. coli strain. (a) HPLC-MS spectrum of 1. (b) MS/MS spectrum of 1 collected at the collision energy of 4.0 eV. Important fragment ions that are assigned (21) are shown in red. (c) ¹H NMR spectrum of 1. (d) ¹H NMR TOCSY spectrum of 1 collected at the mixing time of 100 ms, showing important correlations (numbered in red). 1: δ 0.95-0.75 (Val-CH₃) and 5.15 (Val-Hα); 2: δ 1.15-1.05 (Val-CH₃) and 5.21 (Val-Hα); 3: δ 1.45-1.35 (Ala-CH₃) and 5.00-4.90 (Ala-Hα); 4: δ 1.45-1.35 (Ala-CH₃) and 6.96 (Ala-NH), and δ 1.45-1.35 (Ala-CH₃) and 6.81 (Ala-NH).

FIG. 5. Chemical characteristic spectra of compound 2 produced by the engineered E. coli strain. (a) HPLC-MS spectrum of 2. (b) MS/MS spectra of 2 collected at the collision energy of 3.0 eV, (c) 4.0 eV and (d) 4.5 eV. Important fragment ions in the MS/MS spectra that are assigned (21) are shown in red. (e) ¹H NMR spectrum of 2. (f) ¹H NMR TOCSY spectrum of 2 collected at the mixing time of 100 ms. 1:δ 0.72 (Ala-CH₃) and 5.15-4.90 (Ala-Hα); 2: δ 0.72 (Ala-CH₃) and 8.39 (Ala-NH); 3: δ 1.46 (Ala-CH₃) and 4.85-4.70 (Ala-Hα); 4: δ 1.46 (Ala-CH₃) and 7.21 (Ala-NH); 5: δ 0.87 (Val-CH₃) and 4.27 (Val-Ha); 6: δ 0.87 (Val-CH₃) and 5.22 (Val-Hα); 7; δ 1.06 (Val-CH₃) and 4.27 (Val-Hα); 8: δ 1.06 (Val-CH₃) and 5.22 (Val-Hα); 9:δ 1.10 (Val-CH₃) and 4.27 (Val-Hα), and δ 1.12 (Val-CH₃) and 4.27 (Val-Hα); 10: δ 1.10 (Val-CH₃) and 5.22 (Val-Hα), and δ 1.12 (Val-CH₃) and 5.22 (Val-Hα).

FIG. 6. Quantitative analysis of compound 2 (A) Chromatograms were obtained from a UV detector and (C—F) mass spectrometer. (B) A mass spectrum scanning for masses ranging from m/z 600 to 1500 for retention time (*) 17.55 min reveals two major ions (m/z 1087 and 1109). Each centroid represents compound 2 associated with a proton adduct or sodium adduct, respectively. The mass chromatogram was obtained from 1 mL samples for titer quantitation at each time point according to varying culture conditions: (C) M9 minimal medium with a single dose of QXC, (D) M9 minimal medium with a daily dose of QXC, (E) LB medium with a daily dose of QXC, and (F) M9 minimal medium devoid of QXC dosing.

FIG. 7. Comparison of three 50 mL cultures differing in medium and the method in which the bicyclic starter unit: (♦) MS minimal medium culture with a single dose of QXC at the point of induction; (•) M9 minimal medium culture supplied daily with 5 mg of QXC; and (▪) LB medium culture supplied daily with 5 mg of QXC. The data points are averaged values of three runs.

FIG. 8. Correlation of OD₆₀₀ (♦) and pH (□) of small culture over time: (A) culture in M9 minimal medium furnished with QXC at the point of induction for a final concentration of 0.1 mg/mL; (B) culture in M9 minimal medium furnished daily with 5 mg of QXC; and (C) culture in LB medium furnished daily with 5 mg of QXC. The data points are averaged values of three runs.

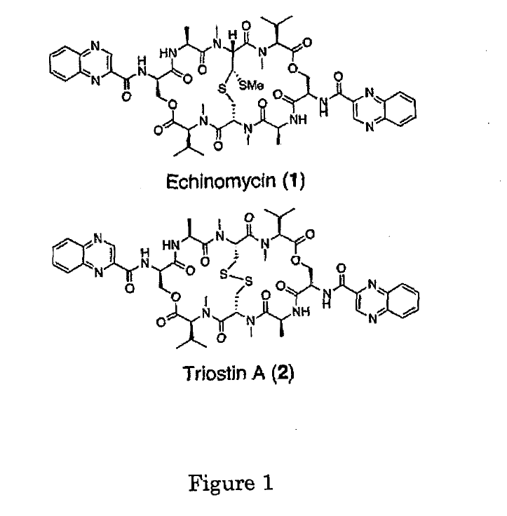

### DETAILED DESCRIPTION OF THE INVENTION

An artillery of natural products as potential drugs or seeds of promising medication have their limitations due to insufficient material for investigative clinical trial. Many soil and marine bacteria are not amenable to cultivation and require time-consuming, highly optimized conditions for mass-production of desired secondary metabolites for clinical and commercial use (22). Therefore, a fast, simple system for heterologous production of natural products is desired.

The present invention demonstrates what the inventors believe to be the first example of the de novo total biosynthesis of biologically active forms of heterologous NRPs in Escherichia coli (E. coli). The system can serve not only as an effective and flexible platform for large-scale preparation of natural products from simple carbon and nitrogen sources, but also as a general tool for detailed characterizations and rapid engineering of biosynthetic pathways for microbial syntheses of novel compounds and their analogs.

Recently, the development of E. coli as a vehicle for heterologous metabolite biosynthesis has seen considerable progress. Perhaps, the greatest advantage of using E. coli is the wealth of knowledge available on its metabolic pathways and genetic make-up, as well as the availability of well-established techniques for its genetic manipulation. Additionally, the ease of E. coli fermentation makes this organism particularly suitable for metabolite overproduction. Its tolerance toward heterologous protein productions and short doubling time are also vital to the efforts. However, the inventors believe no biologically active complex natural product synthesized by heterologous polyketide synthase (PKS), NRP synthetase (NRPS) or mixed PKS/NRPS has been obtained de novo from E. coli. Therefore, the inventors established an E. coli system capable of total biosynthesis of biologically active forms of NRPs.

Compound 1 is an NRP isolated from various bacteria, including Streptomyces lasaliensis (23), that belongs to the large family of quinoxaline antibiotics that have quinoxaline chromophores attached to the C₂-symmetric cyclic depsipeptide core structure. Great interest in this group of compounds stems from its potent antibacterial, anticancer and antiviral activities. Many, including 1 and triostin A 2, exhibit nanomolar potency (24). Also, 1 contains unique chemical structures, including the quinoxaline-2-carboxylic acid (QC) moiety and the thioacetal bridge, whose biosynthetic mechanisms remain unknown.

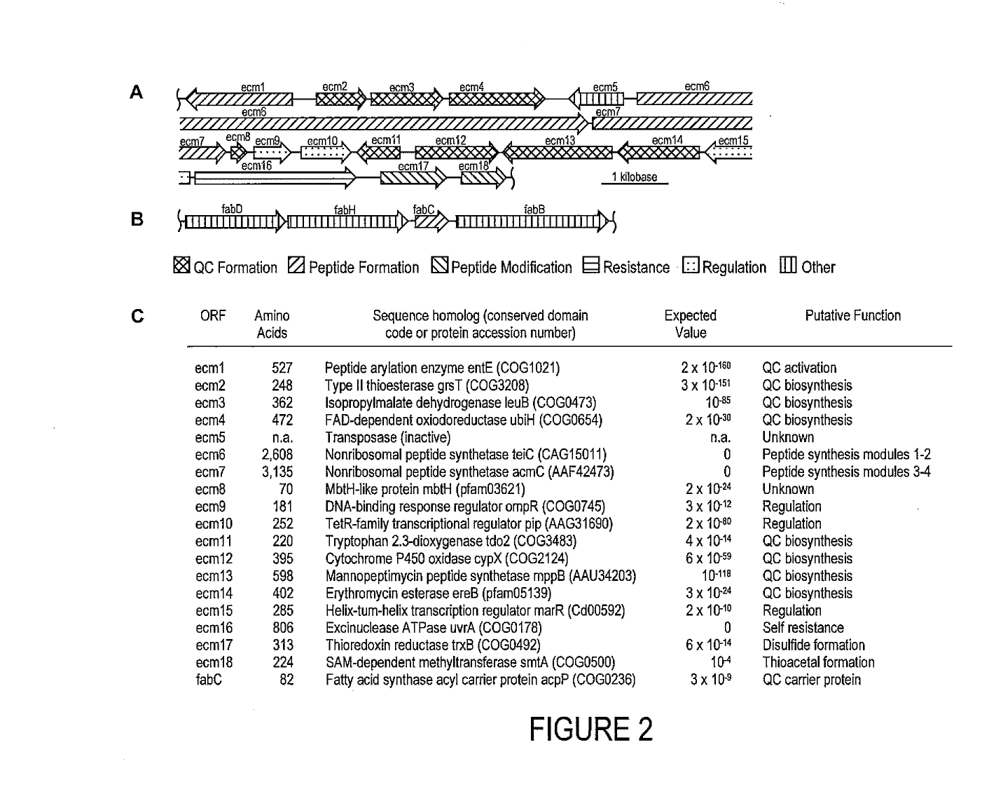

The inventors isolated the echinomycin biosynthetic gene cluster from the S. lasaliensis linear plasmid in accordance with Watanabe, et al. 2006. DNA sequence analysis of the 36 kilobase-long cluster revealed the presence of eight genes that appeared responsible for the QC biosynthesis (ecm2-4, 8, 11-14), five genes for the peptide backbone formation and modifications (ecm 1, 6, 7, 17, 18) and a resistance gene (ecm16) (FIG. 2). Based on the previous finding that L-tryptophan is the precursor for QC (25) and the predicted functions of Ecm2-4, Ecm8 and Ecm11-14, the inventors believed that the QC biosynthesis (Scheme 1a) parallels the first stage of nikkomycin (26) biosynthesis where Ecm12 hydroxylates L-tryptophan bound to the thiolation (T) domain of Ecm13. The product (2S,3S)-β-hydroxytryptophan 3, as determined by substrate feeding experiments (unpublished data), is released from Ecm13 by the thioesterase (TE) activity of Ecm2. Then, as in the first two steps of the kynurenine biosynthesis (27), oxidative ring-opening of 3 by Ecm11 and subsequent hydrolysis by Ecm14 can give β-hydroxykynurenine 4. Subsequently, oxidative cyclization and hydrolysis of 4 by Ecm4 to form N-(2′-aminophenyl)-β-hydroxyaspartic acid 5 and Ecm3 oxidizing 5 to give N-(2′-aminophenyl)-β-ketoaspartic acid 6 can follow. Finally, 6 can undergo spontaneous decarboxylation, cyclic imine formation and oxidative aromatization to give QC.

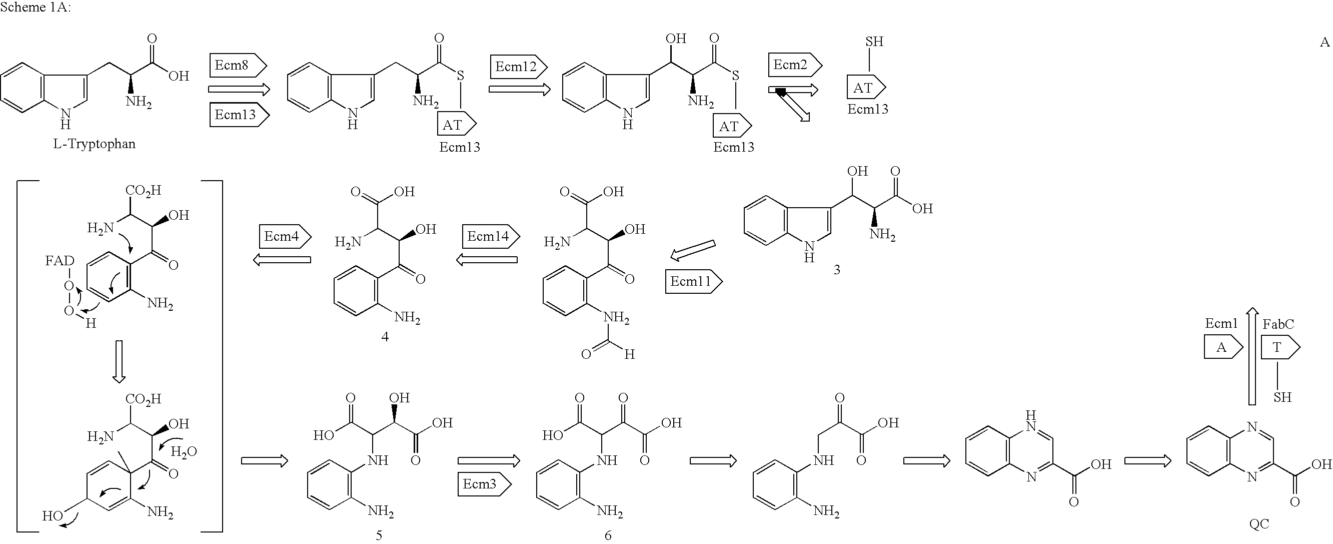

Curiously, the aryl carrier protein (ArCP) required for incorporating QC into 1 was absent from the cluster. However, as in the triostin A biosynthesis (28), the inventors expected the adenylation (A) domain-containing Ecm1 to activate and transfer QC to the phosphopantetheine arm of FabC, the fatty acid biosynthesis acyl carrier protein (ACP). The first module of the bimodular NRPS Ecm6 can accept QC-S-FabC as the starter unit, while Ecm6 and the second NRPS Ecm7 catalyze seventeen chemical reactions for the peptide core formation (Scheme 1b). Ecm7 contains a terminal TE domain that appears to homodimerize and cyclorelease the peptide chain (Scheme 1c)(29). The cyclized product can then become the substrate for an oxidoreductase Ecm17 that can catalyze an oxidation reaction within the reducing cytoplasmic environment to generate the disulfide bond in 2.

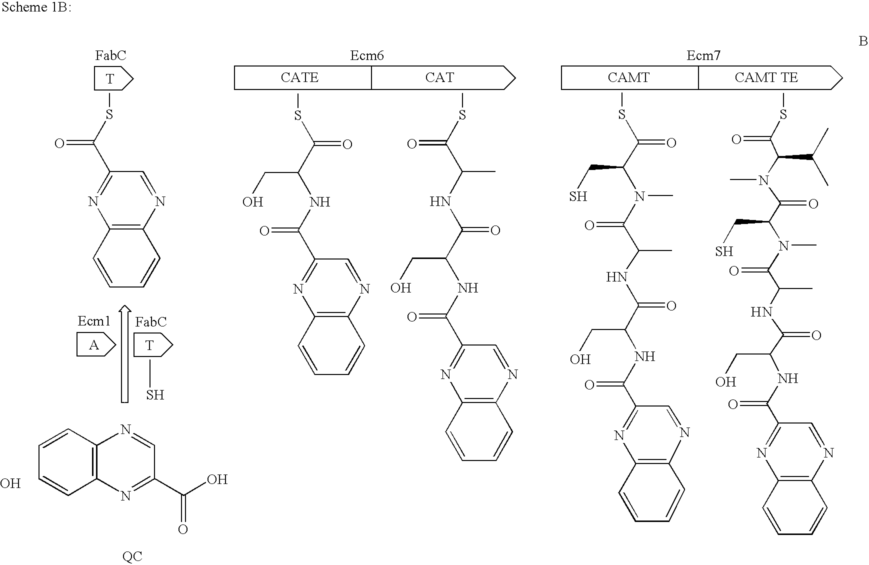

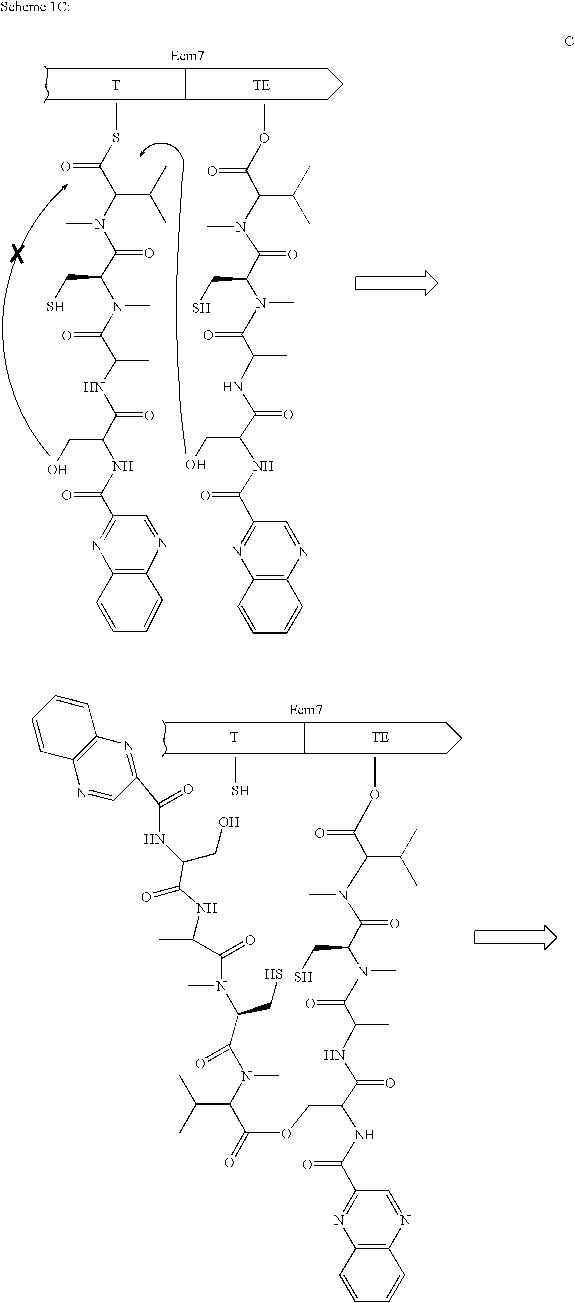

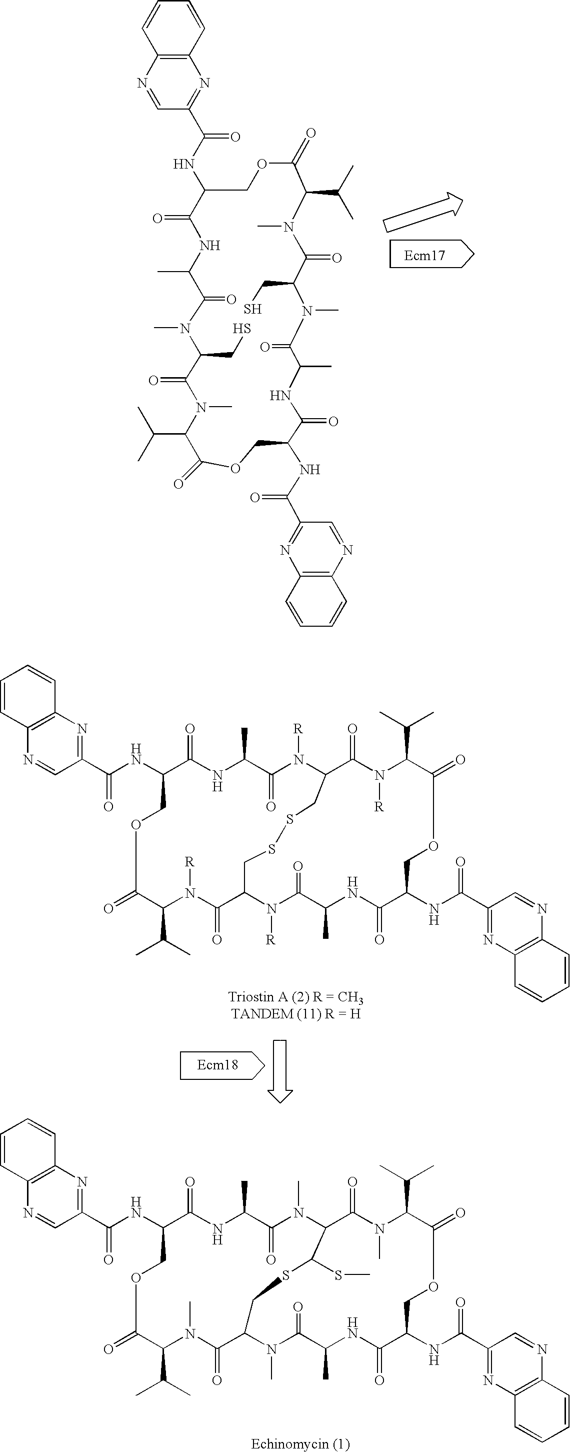

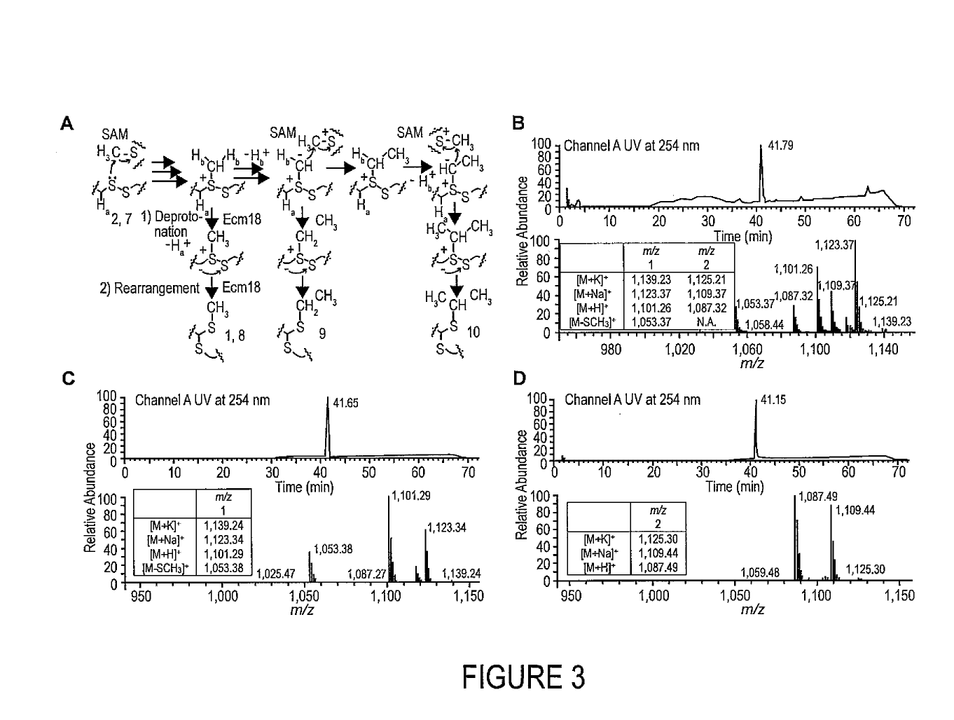

The last step of echinomycin biosynthesis involves an unusual transformation of the disulfide bridge of 2 into a thioacetal bridge (30). Ecm18, which is highly homologous to a known S-adenosyl-L-methionine (SAM)-dependent methyltransferase, is thought to be responsible (FIG. 3a). To verify this, the inventors demonstrated in vitro that purified Ecm18 catalyzes the transformation of 2 to 1 in the presence of SAM (FIG. 3b-d). The inventors believe this is the first finding of a single methyltransferase being responsible for the unprecedented biotransformation of a disulfide bridge into a thioacetal bond. Interestingly, a group of compounds, SW (7, 9)(31) and UK (8, 10) (32) isolated from Streptomyces sp. SNA15896 and S. braegensis, respectively, was found to share the identical backbone while having different modifications at the inter-backbone bridge (Scheme 1d). While the conversion of 7 to 8 can proceed the same way as in the bioconversion of 2 to 1, the formation of 9 and 10 demands attaching alkyl substituents of differing lengths onto the disulfide bridge. Nonetheless, the reaction mechanism via a sulfonium ylide formation involving deprotonation and methyltransfer (33, 34), similar to the mechanism for the Ecm18-catalyzed reaction, can explain a single SAM-dependent methyltransferase performing an iterative methylation of a disulfide bridge and subsequent deprotonation and rearrangement to give 9 and 10 (FIG. 3a). As the inventors observed no further methylation of 1 or thioacetal conversion of a des-N-methyl derivative of 2 called TANDEM 11 (35) by Ecm18, the inventors suspect the presence of another related methyltransferase that can iteratively methylate a disulfide bond. Since the inventors are unaware of methyltransferases capable of performing multiple rounds, the inventors have searched the SNA15896 genome for the 7 and 9 biosynthetic cluster and its associated methyltransferase(s) to obtain further insight into the mechanism of the unique enzymatic thioacetal bridge formation.

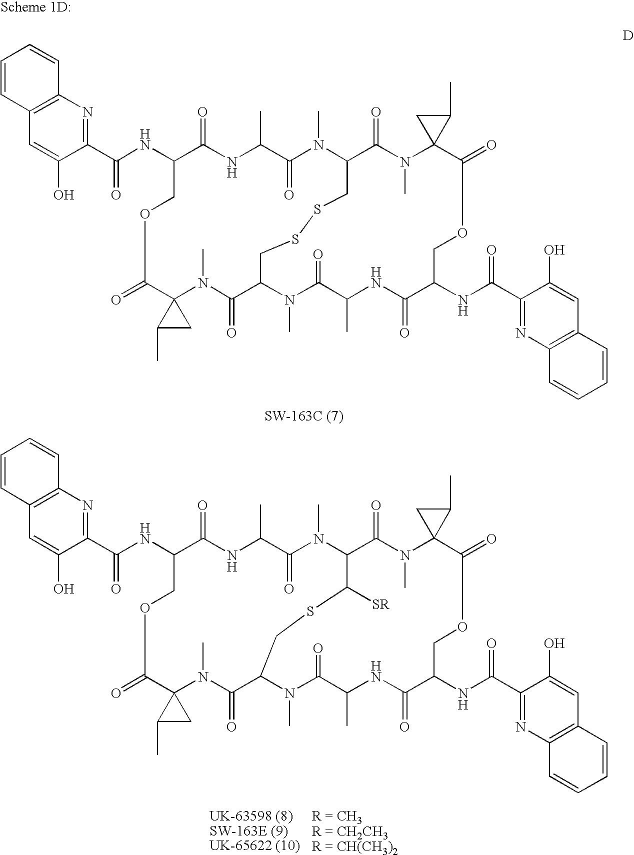

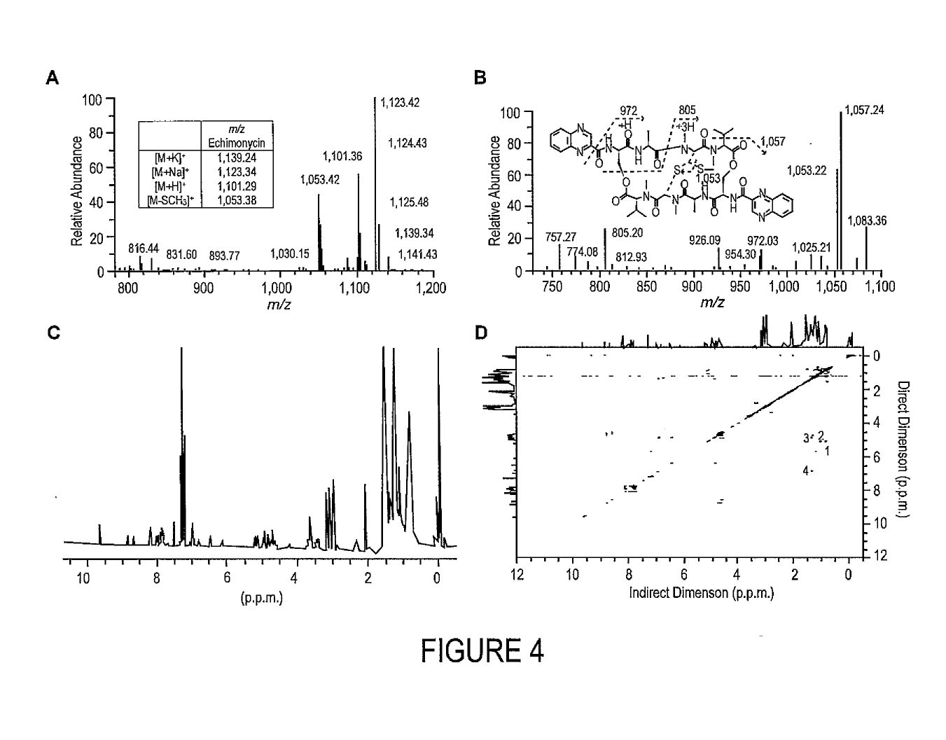

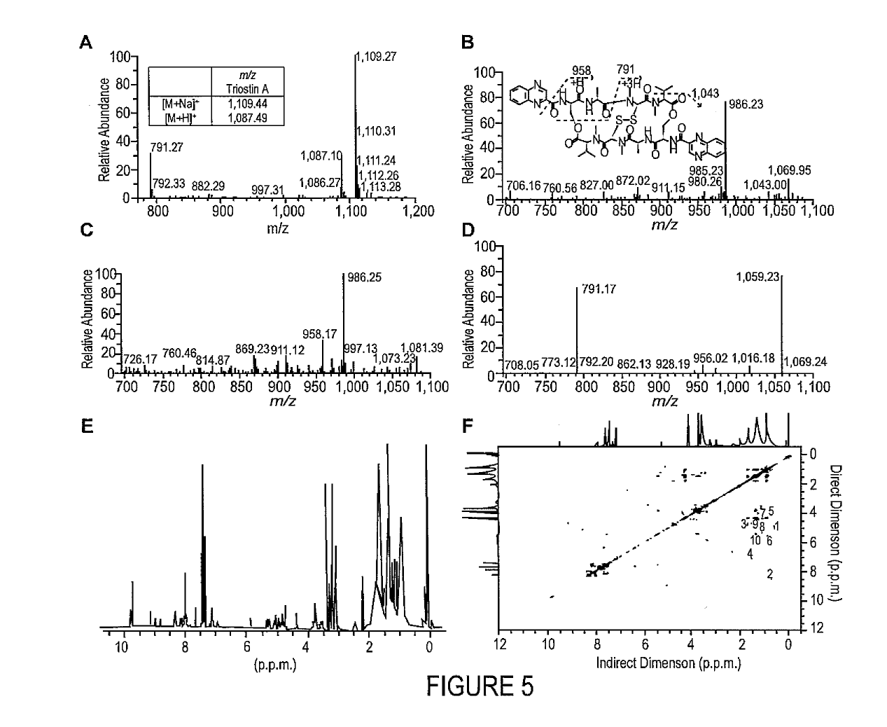

For the E. coli production of 1, after confirming the feasibility of expressing each of the fifteen S. lasaliensis genes (ecm 1-4,6-8, 11-14, 16-18, fabC) in E. coli, the inventors assembled the fifteen genes along with the Bacillus subtilis phosphopantetheine transferase gene sfp, known to efficiently phosphopantetheinylate heterologous ACPs and T domains (36), into three separate plasmids with each gene carrying its own T7 promoter, ribosome binding site and T7 transcriptional terminator (13). The inventors chose this multi-monocistronic arrangement for the multi-gene assembly, not only for simplifying the assembly process but also for minimizing previously observed premature terminations and mRNA degradation in transcribing excessively long polycistronic gene assemblies (37). Moreover, orthogonal origins of replication and antibiotic resistance genes were used to ensure the stable retention of all three plasmids in E. coli (38). The E. coli strain BL21 (DE3) transformed with the three plasmids was subjected to eight-day long fed-batch fermentation in minimal medium. When the culture extract was fractionated by high-performance liquid chromatography (HPLC) and analyzed by electrospray ionization-mass spectrometry (ESI-MS) (FIG. 4a) and MS/MS (FIG. 4b), the data were consistent with the chemical structure of 1. Also, the ¹H nuclear magnetic resonance (NMR) spectrum (FIG. 4c) showed the presence of characteristic resonances at δ 9.66 (s, 1H, QC H-3), 9.64 (s, 1H, QC H-3), and 2.10 (s, 3H, —SCH₃). Additional TOCSY NMR data (FIG. 4d) confirmed unambiguously the E. coli-produced compound as the intact 1. The final yield was 0.3 mg of 1 per liter of culture. These results established the identity of the genes for the biosynthesis of 1. More importantly, however, they serve as what the inventors believe to be the first example of de novo production of a bioactive form of heterologous NRP in E. coli. Furthermore, to demonstrate the ease and effectiveness of modifying the E. coli-based heterologous biosynthetic system, the inventors chose to convert the echinomycin biosynthetic pathway into a triostin A biosynthetic pathway. The inventors modified the plasmid through removal of ecm18. The resulting strain produced the expected compound 2 at a yield of 0.6 mg per liter of culture as confirmed by MS and ¹H NMR, along with the MS/MS profile and the TOCSY spectrum (FIG. 5).

When introducing any exogenous biosynthetic pathway into E. coli, the toxicity of the biosynthetic product can impair the host. This problem can be circumvented by introducing a self-resistance mechanism into E. coli that confer resistance without destroying the product. For echinomycin biosynthesis, the homology between Ecm16 and daunorubicin resistance-conferring factor DrrC (39), and the similarity of the mode of action between 1 and daunorubicin (40) suggested that Ecm16 achieves non-destructive resistance against 1 in S. lasaliensis. Subsequently, the inventors were able to demonstrate that ecm16 conferred echinomycin resistance to BL21 (DE3). Also, when ecm16 was absent from the system, the growth of the host was hampered, suggesting that sufficient amounts of 1 and 2 would have been unattainable without the self-resistance mechanism in place.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### Materials and Methods

Chemicals. Antibiotics were used at the following concentrations: carbenicillin 100 μg/mL, kanamycin 50 μg/mL, and spectinomycin 50 μg/mL. QXC was purchased from Sigma-Aldrich. Other chemical reagents were purchased at the highest commercial quality and used without further purification.

Bacteria Strains and Media. Common procedures, including plasmid manipulation, transformation, and other standard molecular biological techniques, were carried out as previously described in Sambrook and Russell (41). E. coli DH5 cc purchased from Invitrogen was used for plasmid amplification and grown in Luria-Bertani (LB) medium or M9 minimal medium (41) at 37° C. Overproduction of recombinant proteins for production of compound 2 was carried out in E. coli BL21 (DE3) (Invitrogen). Cell growth was monitored using optical density and measured at 600 nm (OD₆₀₀), and isopropyl 1-thio-β-D-galactopyranoside (IPTG) was used to induce. Feed media used for fermentation contained 430 g/L of glucose, 3.90 g/L of MgSO₄, 10 g/L of alanine, trace metal (0.278 g/L of FeCl₃.6H₂O, 0.130 g/L of ZnCl₂, 0.013 g/L of CaCl₂.2H₂O, 0.021 g/L of NaMoO₄.2H₂O, 0.190 g/L of CuSO₄.5H₂O, 0.024 g/L of H₃BO₃), and vitamins (0.00420 g/L of riboflavin, 0.05456 g/L of pantothenic acid, 0.06078 g/L of nicotinic acid, 0.014140 g/L of pyridoxin, 0.00062 g/L of biotin, and 0.00048 g/L of folic acid).

Assembling the plasmid-based echinomycin biosynthetic gene cluster. Initially, each ORF was cloned individually into pET28b (Novagen)-derived pKW409 as either a Nde I-EcoR I or a Nde I-Xho I fragment prepared by PCR (13). In pKW409, the single Xba I recognition site was moved to the 5′ side of the T7 promoter, and a Spe I recognition site was created at the 3′ side of the T7 terminator. The assembly formation exploited the compatibility of the cohesive ends generated by Xba I and Spe I digestion. The cassette arrangement not only facilitated evaluation of the expression level of each gene individually, but also was necessary for rapid construction of the multi-monocistronic gene assemblies (13).

Thioacetal formation assay. The assay mixture containing 10 μM Ecm 18 and 10 mM SAM in 0.1 M Tris-HCl pH 7.2 was pre-incubated at 30° C. for 5 min. After addition of 2 to the final concentration of 1 mM, the reaction was run at 30° C. for five minutes before being terminated by addition of 10% (w/v) SDS. The reaction mixture was extracted with ethyl acetate, and the extract was concentrated in vacuo to give a white residue. Compound 1 and 2 were isolated from the extracts using PTLC (5% MeOH/CHCl₃). The isolated samples of 1 and 2, along with the mixture were analyzed independently by HPLC-MS Alltech 2.1×100 mm C₁₈ reverse-phase column. Samples were subjected to a linear gradient of 5 to 95% CH₃CN (v/v) in H₂O supplemented with 0.05% (v/v) formic acid over 60 min at a flow rate of 0.15 ml/min at room temperature. Compound 1 eluted at 41.65 min under the condition described above, and gave characteristic ions at m/z=1139.24 [M+K]⁺, 1123.34 [M+Na]⁺, 1101.29 [M+H]⁺ and 1053.38 [M-SCH₃]⁺. Similarly, 2 eluted at 41.15 min and gave characteristic ions at m/z=1125.30 [+K]⁺, 1109.44 [M+Na]+ and 1087.49 [M+H]⁺. These results matched well with the analyses of the authentic samples of 1 and 2 (FIG. 3d).

E. coli production of 1 and 2. BL21 (DE3) transformed with pKW532/pKW538/pKW541 and pKW532/pKW539/pKW541 for the production of 1 and 2, respectively, was incubated at 37° C. overnight in 2 ml Luria-Bertani (LB) medium and subsequently in 100 ml M9 minimal medium. The entire culture was used to inoculate 1.5 liters of M9 minimal medium kept at 37° C., pH 7.0 by the BioFlo110 fermentor system (New Brunswick Scientific). Once the glucose was exhausted from the medium as indicated by a sudden increase of the dissolved oxygen level, feeding of the feed media (42) was initiated. When the culture reached an OD₆₀₀ of 11, the temperature was reduced to 15° C., and isopropylthio-β-D-galactoside (IPTG) was added to the final concentration of 200 μM. After eight to twelve days of incubation, the culture was centrifuged to separate the supernatant and the cells. The supernatant and the cell pellet were extracted with ethyl acetate and acetone, respectively. The extracts were combined and concentrated in vacuo to give an oily residue, which was fractionated by silica gel flash column chromatography with 50% MeOH/CHCl₃. The fractions containing the target compound were collected and further purified by a series of preparative thin-layer chromatography (PTLC: i. 50% EtOAc/hexane; ii. 2-butanone; iii. 5% MeOH/CHCl₃) to afford purified samples 1(43) and 2(44). The isolated compounds were fully characterized by HPLC-MS, MS/MS, ¹H NMR and ¹H NMR TOCSY.

Echinomycin resistance assay. E. coli echinomycin resistance was determined using BL21 (DE3) transformed with pKW409 carrying ecm 16. The transformant was incubated in 3 ml LB medium at 37° C. for 5 hours. The culture was spread on LB agar plates containing two different concentrations of echinomycin (10 and 100 μg/ml) supplemented either with or without 300 μM IPTG. The plates were incubated at 37+C overnight to determine colony formation. Accession codes. The echinomycin biosynthetic gene cluster sequence has been deposited in DNA Data Bank of Japan with the accession numbers AB211309 and AB211310.

For de novo Production of Compound 2 in E. coli Using Shake Flask. Three plasmids, pKW532 (ecm2-4, 8, and 11-14), pKW539 (ecm1, 16, 17, fabC, and sfp) and pKW541 (ecm6 and 7) were transformed into E. coli BL21 (DE3) (13). The cells were incubated at 37° C. overnight in 2 mL of LB medium supplemented with carbenicillin, spectinomycin, and kanamycin. Subsequently, 0.5 mL of the culture was used to inoculate 50 mL of M9 minimal medium with the antibiotics described above. The culture was grown at 37° C. until its OD₆₀₀ reached 0.3-0.6, at this point, the temperature was reduced to 15° C. and IPTG was added at a final concentration of 200 μM. Simultaneously, 10 mL of a feed medium (11) was added and continuously shaken at 150 rpm for 8 days.

For QXC Fed Production of Compound 2 in E. coli Using Shake Flask. Two transformants were prepared: a culture transformed with three plasmids pKW532/pKW539/pKW541 and another transformed with only two plasmids pKW539 and pKW541, the biosynthetic gene cluster for compound 2 (45, 46). The described constructs were transformed into BL21 (DE3) and cultivated for production of 2. Culture conditions for BL21 (DE3) transformed with pKW532/pKW539/pKW541 are the same as those used for de novo production. Two feed methods were explored for cells transformed with pKW532/pKW539/pKW541. A single dose of QXC was added to our culture at the point of gene expression for a final concentration of 0.1 mg/mL. Subsequent experiments where a daily dose of QXC at 5 mg per feed initiated at the point of gene expression were also examined. Production of 2 in LB medium under the same culture conditions with a daily supply at 5 mg of QXC per feed for cultures transformed with pKW532/pKW539/pKW541 was also explored. BL21 (DES) transformed with pKW539/pKW541 was incubated at 37° C. overnight in 2 mL of LB medium supplemented with spectinomycin and kanamycin. Subsequently, 0.5 mL of the culture was used to inoculate 50 mL of M9 minimal medium with added antibiotics as described above. All other culture conditions matched those described for de novo production of 2. A daily feed of QXC in the amount of 5 mg per feed was initiated at the induction point.

Quantitative Analysis of Compound 2 Production. Upon induction of gene expression, 1 mL of the culture was collected and centrifuged to pellet the cell and harvest the supernatant for analysis. The supernatant was extracted with ethyl acetate (2×1 mL) and concentrated in vacuo. The resultant residue was dissolved with 100 μL of methanol and 15 μL of the resulting mixture was analyzed by LC-MS using an Alltech 2.1×100 mm C₁₈ reverse-phase column. Samples were separated on a linear gradient of 5% to 95% CH₃CN (v/v) in H₂O supplemented with 0.05% (v/v) formic acid over 40 min at room temperature and a flow rate of 0.1 mL/min. To quantitate the production of 2, a Finnigan LCQ Deca XP mass spectrometer equipped with an electrospray probe operating on positive mode was used. We prepared a standard curve with a range between 1 ng and 10 μg of reference compound 2. Mass spectra collected in single ion monitoring mode were obtained by monitoring two ions ([M+H]⁺ and [M+Na]⁺, m/z 1087 and 1109, respectively) observed at retention time 17.5 min of bp070298yt00001 the LC. The following optimized values were employed for data acquisition: capillary temperature 275° C.; spray voltage 5 kV; source current 80 μA; capillary voltage 12 V; sheath gas N₂ flow 60 (arbitrary units). Accumulated compound 2 in culture was then extrapolated using our standard curve following the steps described above.

### Results and Discussion

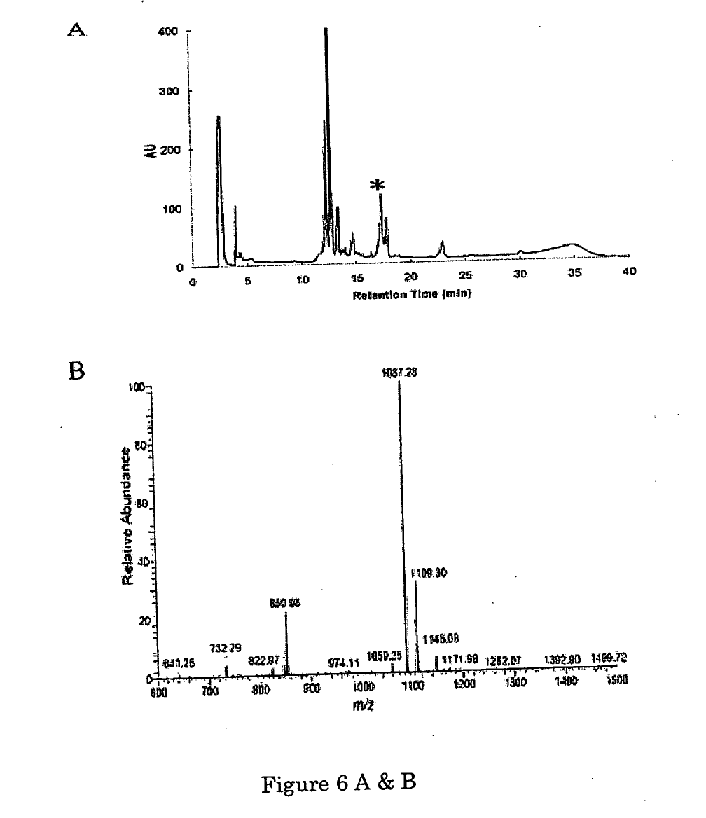

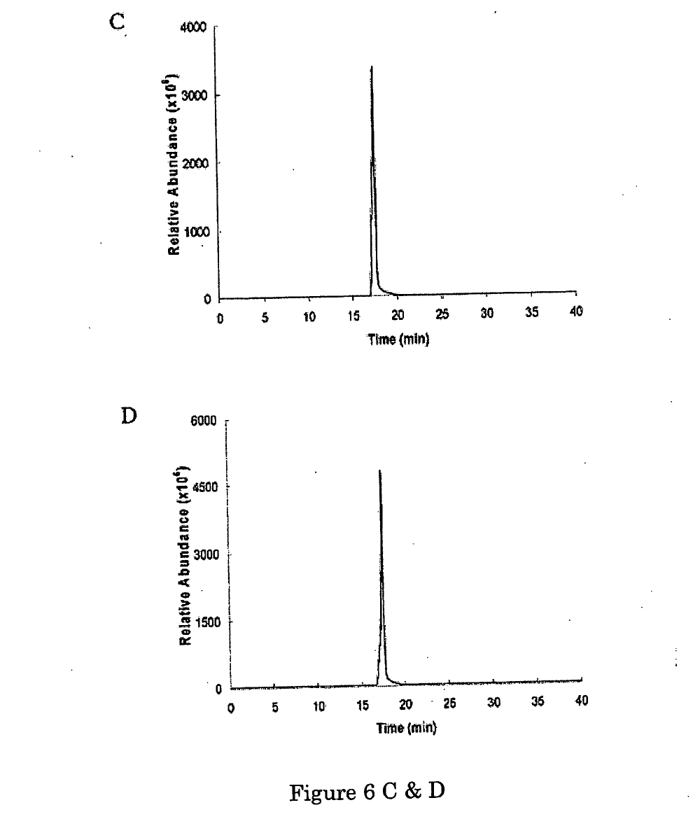

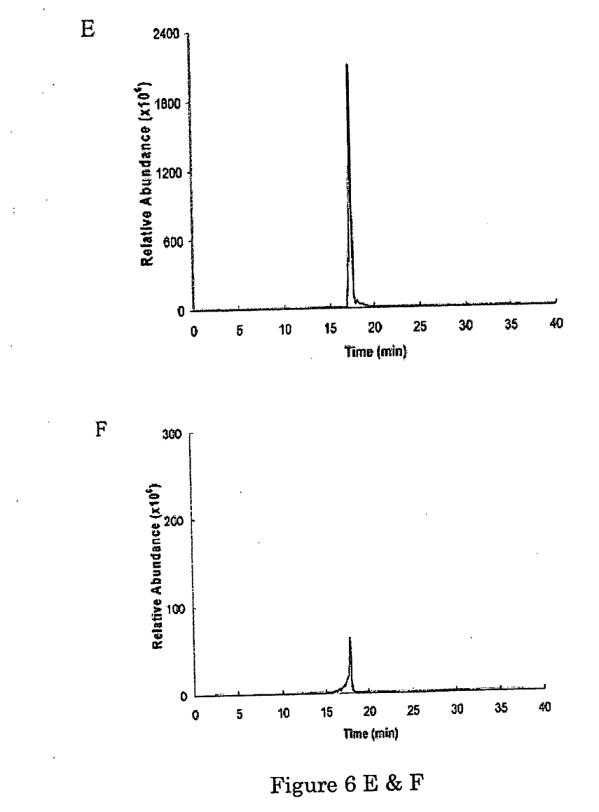

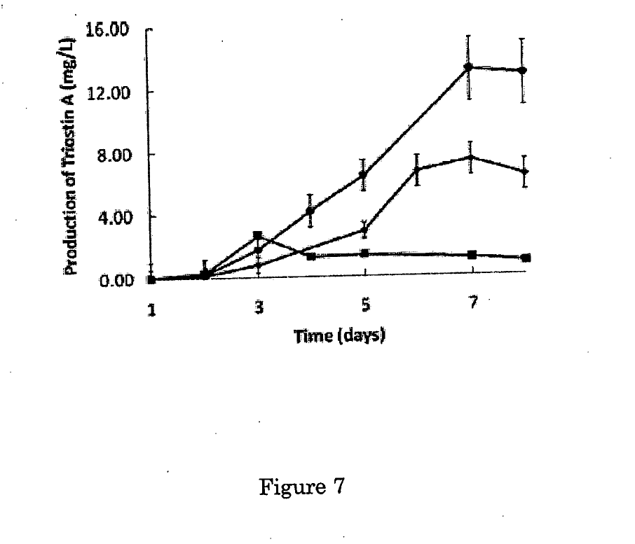

In our previous report, E. coli expression of a multiple-plasmid system in M9 minimal medium containing the complete biosynthetic pathway of 2 produced 0.6 mg/L of isolated compound via fed-batch fermentation. Although de novo production of NRP, compound 2, was a successful achievement, its titer was modest in amount despite the use of the fed-batch fermentation process (Table 1). To address the low productivity by means of a simple and quick procedure, we analyzed the production of 2 using small-scale shake flask culture (FIG. 6). Levels of compound 2 were deplorable for de novo production while using shake flask and M9 medium reaching a meager titer of only 0.1 mg/L (FIG. 6F, 7). On the basis of past experiments, our titer assessment begins with focus on the availability of the speculative starting unit, QXC. Levels of the bicyclic chromophore were miniscule and undetectable when its intact biosynthetic pathway was independently expressed in E. coli. Suspecting this step as the encumbrance for desirable antibiotic production, we designed three experiments to corroborate our suspicion. First, our culture transformed with only two plasmids exclusive of the QXC biosynthetic gene cluster (pKW532) was given a daily supply of QXC. Second, a culture transformed with pKW532/pKW539/pKW541 was given a single dose of QXC. Last, a culture transformed with pKW532/pKW539/pKW541 was supplied daily with QXC. The first scenario abolished production of 2. Our second scenario conferred a maximal titer of 7 mg/L of 2 (FIGS. 6C and 7). Surprisingly, our third experiment where QXC was supplied daily to bacterium transformed with our triple-plasmid system during the point of gene expression produced a notably higher titer of 2 reaching a threshold concentration of 13 mg/L (FIGS. 6D and 7). Moreover, we evaluated the productivity dependence on medium by comparing NRP production in M9 minimal medium versus LB medium under similar conditions described for day by day feeding of QXC. LB medium provided subpar titer of compound 2, reaching maximal concentration of 3 mg/L after 3 days of incubation postinduction (FIGS. 2E and 3). In terms of shake flask experiments, transformants carrying the complete biosynthetic pathway of compound 2 with a daily supply of QXC can produce a titer that is elevated more than 130-fold relative to de novo production of the antibiotic. Noteworthy production under continuous feed of QXC revealed a titer approximately 22-fold beyond that of de novo fed-batch fermentation. On the basis of these observations, the intact biosynthetic pathway of 2 including QXC's gene cluster when transformed into E. coli readily accepted QXC as the starting unit. However, exclusion of QXC's biosynthetic genes, ecm2, ecm3, ecm4, ecm8, ecm11, ecm12, ecm13, and ecm14 (Scheme 1), our heterologous host was not able to provide measurable levels of 2 even with exogenous QXC.

TABLE 1
Conditions for Maximal Production of Compound 2

| | shake flask^{a} | | fed-batch fermentor |
|---|---|---|---|
| | M9 | LB | M9 |
| daily feed^{b} | 13^{c} | 3 | NA |
| single dose | 7 | NA | NA |
| de novo | 0.1 | NA | 0.6^{d} |

^{a}Culture vessels are described in Materials and Methods.
^{b}Daily feed of QXC was supplied at 5 mg/day post induction until harvest of culture. A single dose of QXC was supplied once at the same amount during the point of induction. QXC was not supplied for de novo production of compound 2.
^{c}The reported level of compound 2 is shown in milligrams of product per liter of culture.
^{d}Isolated quantity of 2 from fed-batch fermentation.¹³
NA, not applicable;
M9, M9 minimal medium;
LB, Luria-Bertani medium.

These findings suggest an indispensable interaction between QXC's biosynthetic protein or proteins with either ecm1 or ecm1 and fab C. Improving modest yields from de novo production of 2 by simply adding commercial QXC has confirmed its assembly as the bottleneck. These findings have provided further evidence and narrowed QXC's role as the priming unit for biosytnthesis of 2+Facile expression of the intact biosynthetic gene cluster for compound 2 in a heterologous host, E. coli has eased our attempts at garnering data to provide a more comprehensive picture of 2's dependence on QXC. Although we have identified and presented the stereochemical assignment for the β-hydroxytryptophan intermediate in the QXC biosynthetic pathway (19), it will require additional investigation in order to aid and circumvent the inherent challenges of biological studies and metabolic engineering for de novo production of 2 in E. coli to ultimately increase its yield. Further elucidation of the pathway is ongoing.

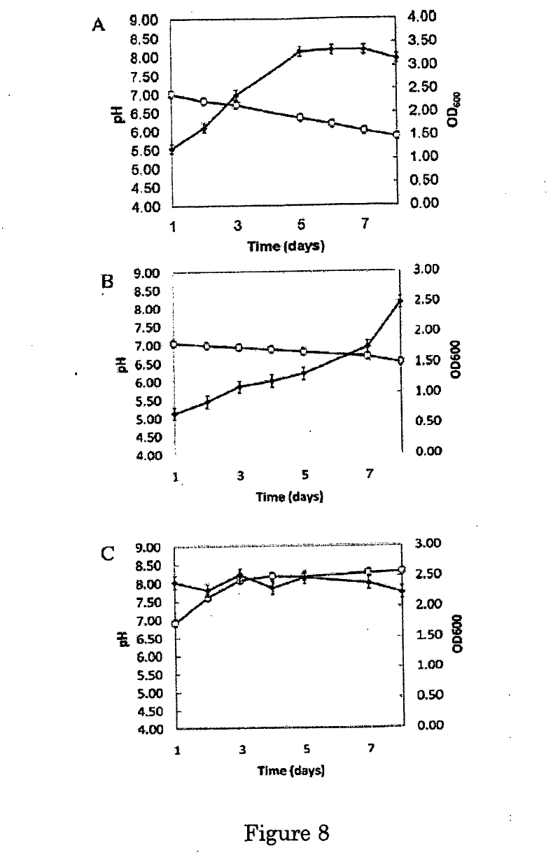

A pH and cell growth profile is shown in FIG. 8. Negligible influence on culture pH was observed even when QXC, an acidic compound, was supplemented daily. Interestingly, using LB medium for cultivation of our recombinant E. coli instigated an experimental spike in pH reaching its peak on the third day following induction. Illustrated in FIG. 4C, productivity of 2 was thwarted and residual antibiotic was degraded as our culture was allowed to continue. This observation was also documented for production of echinomycin (1) by S. echinatus, the original producing host of compound 1 (47). In contrast, when using M9 minimal medium, such correlation between compound productivity and its pH profile was veiled unlike cultures grown in LB medium.

Mounting evidence has revealed how fed-batch fermentation technology for metabolic engineering may possess the capability of producing a more substantial level of 2 by merely supplementing exogenous starting unit during biosynthesis of compound 2. Equally, testing of precursor-directed biosynthesis by means of feeding an assortment of chromophore to the heterologous expression system may provide us with a combinatorialy engineered biosynthesis of unnatural quinomycin antibiotics. This tolerance is indispensable to the biosynthetic research field because of its simplicity and speedy assembly line.

### CONCLUSIONS

In conclusion, the present invention demonstrates, for what the inventors believe to be the first time, the viability of E. coli-based total biosynthesis of a bioactive form of heterologous complex NRPs from simple carbon and nitrogen sources, paving the way to developing an economical, general platform for one-pot mass-production of natural products and their analogs. The system shows that using a multi-plasmid, multi-monocistronic gene assembly is a straightforward, highly stable and easily modifiable approach for establishing and engineering exogenous biosynthetic pathways in E. coli. With the use of appropriate orthogonal selection markers and origins of replication, in combination with other potential approaches, such as chromosome integration (45), introducing even larger, more complex biosynthetic pathways seems attainable. Combining these current efforts with the successes in introducing other PKS(46,48) and mixed PKS-NRPS(42) pathways into E. coli and engineering of PKS(49) and NRPS(50) should help broaden the scope of E. coli-based heterologous mass-production of a wide range of natural products and their analogs.

The present invention also substantiated QXC as the primer unit of 2 by using a heterologous host for its biosynthesis while supplying the culture with the chromophore. Under optimal conditions, the cultures were then subsidized with QXC to afford 13 mg/L of compound 2, an increase of more than 130-fold relative to its de novo production in shake flask fermentation. By using LC-MS to analyze small-scale culture, the inventors obtained indispensable information in a much shorter time frame. The disclosed method has considerable importance for potential endeavors to creating a quinomycin antibiotic library.

Obviously, many modifications and variation of the invention as hereinbefore set forth can be made without departing from the spirit and scope thereof and therefore only such limitations should be imposed as are indicated by the appended claims.

All patent and literature references cited in the present specification are hereby incorporated by reference in their entirety.

### REFERENCES

The following references are cited herein. The entire disclosure of each reference is relied upon and incorporated by reference herein.
1. Schwarzer, D.; Finking, R.; Marahiel, M. A. Nonribosomal peptides: From genes to products. Nat. Prod. Rep. 2003, 20, 275-287.
2. Shuler, M. L. Bioreactor engineering as an enabling technology to tap biodiversity. The case of taxol. Ann. N.Y. Acad. Sci. 1994, 745, 455-461.
3. Steinerova, N.; Lipavska, H.; Stajner, K.; Caslavska, J.; Blumauerova, M.; Cudlin, J.; Vanek, Z. Production of quinomycin A in Streptomyces lasaliensLs. Folia Microbiol. (Praha) 1987, 32, 1-5.
4. Boger, D. L.; Ichikawa, S.; Tse, W. C.; Hedrick, M. P.; Jin, Q. Total syntheses of thiocoraline and BE-22179 and assessment of their DNA binding and biological properties. J. Am. Chem. Soc. 2001, 123, 561-568.
5. Cane, D. E.; Walsh, C. T.; Khosla, C. Harnessing the biosynthetic code: combinations, permutations, and mutations. Science 1998, 282, 63-68.
6. Marahiel, M. A.; Stachelhaus, T.; Mootz, H. D. Modular peptide synthetases involved in nonribosomal peptide synthesis. Chem. Rev. 1997, 97, 2651-2674.
7. Schwarzer, D.; Finking, R.; Marahiel, M. A. Nonribosomal peptides: from genes to products. Nat. Prod. Rep. 2003, 20, 275-287.
8. Kieser, T.; Bibb, M. J.; Buttner, M. J.; Chater, K. F.; Hopwood, D. A. Practical Streptomyces Genetics; The John Innes Foundation: Norwich, England, 2000.
9. Watanabe, K.; Oikawa, H. Robust platform for de novo productions of heterologous polyketides and nonribosomal peptides in Escherichia coli. Org. Biomol. Chem. 2007, 5, 593-602.
10. Pfeifer, B. A.; Admiraal, S. J.; Gramajo, H.; Cane, D. E.; Khosla, C. Biosynthesis of complex polyketides in a metabolically engineered strain of E. coli. Science 2001, 291, 1790-1792.
11. Pfeifer, B. A.; Wang, C. C.; Walsh, C. T.; Khosla, C. Biosynthesis of yersiniabactin, a complex polyketide-nonribosomal peptide, using Escherichia coli as a heterologous host. Appl. Environ. Microbiol. 2003, 69, 6698-6702.
12. Watanabe, K.; Rude, M. A.; Walsh, C. T.; Khosla, C. Engineered biosynthesis of an ansamycin polyketide precursor in Escherichia coli. Proc. Natl. Acad. Sci. U.S.A. 2003, 100, 9774-9778.
13. Watanabe, K.; Hotta, K.; Praseuth, A. P.; Koketsu, K.; Migita, A.; Boddy, C. N.; Wang, C. C. C.; Oguri, H.; Oikawa, H. Total biosynthesis of antitumor nonribosomal peptides in Escherichia coli. Nature Chem. Biol. 2006, 2, 423-428.
14. Wang, A. H.; Ughetto, G.; Quigley, G. J.; Hakoshima, T.; van der Marel, G. A.; van Boom, J. H.; Rich, A. The molecular structure of a DNA-triostin A complex. Science 1984, 225, 1115-1121.
15. Chen, H.; Patel, D. J. Solution structure of a quinomycin bisintercalator-DNA complex. J. Mol. Biol. 1995, 246, 164-79.
16. Neidle, S.; Pearl, L. H.; Skelly, J. V. DNA structure and perturbation by drug binding. Biochem. J. 1987, 243, 1-13.
17. Furuya, K.; Hutchinson, C. R. The DrrC protein of Streptomyces peucetius, a UvrA-like protein, is a DNA-binding protein whose gene is induced by daunorubicin. FEMS Microbiol. Lett. 1998, 168, 243-249.
18. Reid, D. G.; Doddrell, D. M.; Williams, D. H.; Fox, K. R. A ¹⁵N nuclear magnetic resonance study of the biosynthesis of quinoxaline antibiotics. Biochim. Biophys. Acta 1984, 798, 111-114.
19. Koketsu, K.; Oguri, H.; Watanabe, K.; Oikawa, H. Identification and stereochemical assignment of the β-hydroxytryptophan intermediate in the echinomycin biosynthetic pathway. Org. Lett. 2006, 8, 4719-4722.
20. McGinnis, S. & Madden, T. L. BLAST: At the core of a powerful and diverse set of sequence analysis tools. Nucleic Acids Res. 32, W20-25 (2004).
21. Dell, A. et al. Structure revision of the antibiotic echinomycin. J. Am. Chem. Soc. 97, 2497-2502 (1975).
22. Shuler, M. L. Bioreactor engineering as an enabling technology to tap biodiversity. The case of taxol. Ann N Y Acad Sci 745, 455-461 (1994).
23. Steinerova, N. et al. Production of quinomycin A in Streptomyces lasaliensis. Folia Microbiol. (Praha) 32, 1-5 (1987).
24. Boger, D. L., Ichikawa, S., Tse, W. C., Hedrick, M. P. & Jin, Q. Total syntheses of thiocoraline and BE-22179 and assessment of their DNA binding and biological properties. J. Am. Chem. Soc. 123, 561-568 (2001).
25. Reid, D. G., Doddrell, D. M., Williams, D. H. & Fox, K. R. A 15N nuclear magnetic resonance study of the biosynthesis of quinoxaline antibiotics. Biochim. Biophys. Acta 798, 111-114 (1984).
26. Chen, H., Hubbard, B. K., O'Connor, S. E. & Walsh, C. T. Formation of beta-hydroxy histidine in the biosynthesis of nikkomycin antibiotics. Chem. Biol. 9, 103-112 (2002).
27. Kurnasov, O. et al. Aerobic tryptophan degradation pathway in bacteria: novel kynurenine formamidase. FEMS Microbiol. Lett. 227, 219-227 (2003).
28. Schmoock, G. et al. Functional cross-talk between fatty acid synthesis and nonribosomal peptide synthesis in quinoxaline antibiotic-producing streptomycetes. J, Biol. Chem. 280, 4339-4349 (2005).
29. Trauger, J. W., Kohli, R. M., Mootz, H. D., Marahiel, M. A. & Walsh, C. T. Peptide cyclization catalysed by the thioesterase domain of tyrocidine synthetase. Nature 407, 215-218 (2000).
30. Cornish, A., Waring, M. J. & Nolan, R. D. Conversion of triostins to quinomycins by protoplasts of Streptomyces echinatus. J. Antibiot. (Tokyo) 36, 1664-1670 (1983).
31. Takahashi, K., Koshino, H., Esumi, Y., Tsuda, E. & Kurosawa, K. SW-163C and E, novel antitumor depsipeptides produced by Streptomyces sp. II. Structure elucidation. J. Antibiot. (Tokyo) 54, 622-627. (2001).
32. Rance, M. J. et al. UK-63,052 complex, new quinomycin antibiotics from Streptomyces braegensis subsp. japonicus; taxonomy, fermentation, isolation, characterisation and antimicrobial activity. J. Antibiot. (Tokyo) 42, 206-217. (1989).
33. Barrett, A. G. M., Hamprecht, D., White, A. J. P. & Williams, D. J. Iterative cyclopropanation: A concise strategy for the total synthesis of the hexacyclopropane cholesteryl ester transfer protein inhibitor U-106305. J. Am. Chem. Soc. 119, 8608-8615 (1997).
34. Van Lanen, S. G. & Iwata-Reuyl, D. Kinetic mechanism of the tRNA-modifying enzyme S-adenosylmethionine:tRNA ribosyltransferase-isomerase (QueA). Biochemistry 42, 5312-5320 (2003).
35. Ciardelli, T. L., Chakravarty, P. K. & Olsen, R. K. Des-N-tetramethyltriostin A and bis-L-seryldes-N-tetramethyltriostin A, synthetic analogues of the quinoxaline antibiotics. J. Am. Chem. Soc. 100, 7684-7690 (1978).
36. Lambalot, R. H. et al. A new enzyme superfamily—the phosphopantetheinyl transferases. Chem. Biol. 3, 923-936 (1996).
37. Sørensen, H. P. & Mortensen, K. K. Advanced genetic strategies for recombinant protein expression in Escherichia coli. J. Biotechnol. 115, 113-128 (2005).
38. Lau, J., Tran, C., Licari, P. & Galazzo, J. Development of a high cell-density fed-batch bioprocess for the heterologous production of 6-deoxyerythronolide B in Escherichia coli. J. Biotechnol. 110, 95-103 (2004).
39. Furuya, K. & Hutchinson, C. R. The DrrC protein of Streptomyces peucetius, a UvrA-like protein, is a DNA-binding protein whose gene is induced by daunorubicin. FEMS Microbiol. Lett. 168, 243-249 (1998).
40. Neidle, S., Pearl, L. H. & Skelly, J. V. DNA structure and perturbation by drug binding. Biochem. J. 243, 1-13 (1987).
41. Sambrook, J.; Russell, D. W. Molecular Cloning, 3rd ed.; Cold Spring Harbor Laboratory Press: New York, 2001.
42. Pfeifer, B. A., Wang, C. C., Walsh, C. T. & Khosla, C. Biosynthesis of yersiniabactin, a complex polyketide-nonribosomal peptide, using Escherichia coli as a heterologous host. Appl. Environ. Microbiol. 69, 6698-6702 (2003).
43. Cheung, H. T. et al. The conformation of echinomycin in solution. J. Am. Chem. Soc. 100, 46-54 (1978).
44. Blake, T. J., Kalman, J. R. & Williams, D. H. Two symmetrical conformations of the triostin antibiotics in solution. Tetrahedron Lett. 18, 2621-2624 (1977).
45. Royo, J. L., Moreno-Ruiz, E., Cebolla, A. & Santero, E. Stable long-term indigo production by overexpression of dioxygenase genes using a chromosomal integrated cascade expression circuit. J. Biotechnol. 116, 113-124 (2005).
46. Watanabe, K., Rude, M. A., Walsh, C. T. & Khosla, C. Engineered biosynthesis of an ansamycin polyketide precursor in Escherichia coli. Proc. Natl. Acad. Sci. USA 100, 9774-9778 (2003).
47. Gauvreau, D.; Waring, M. J.; Directed biosynthesis of novel derivatives of echinomycin by Streptomyces echinatus. I. Effect of exogenous analogues of quinoxaline-2-carboxylic acid on the fermentation. Can. J. Microbiol. 1984, 30, 439-450.
48. Peiru, S., Menzella, H. G., Rodriguez, E., Carney, J. & Gramajo, H. Production of the potent antibacterial polyketide erythromycin C in Escherichia coli. Appl. Environ. Microbiol. 71, 2539-2547 (2005).
49. Watanabe, K., Wang, C. C., Boddy, C. N., Cane, D. E. & Khosla, C. Understanding substrate specificity of polyketide synthase modules by generating hybrid multimodular synthases. J. Biol. Chem. 278, 42020-42026 (2003).
50. Gruenewald, S., Mootz, H. D., Stehmeier, P. & Stachelhaus, T. In vivo production of artificial nonribosomal peptide products in the heterologous host Escherichia coli. Appl. Environ. Microbiol. 70, 3282-3291 (2004).

What is claimed is:

1. A method of producing biologically active forms of nonribosomal peptides (NRPs) in Escherichia coli (E. coli) comprising (a) culturing an E. coli bacterium which has an ability to produce NRPs in a medium, and (b) collecting the target NRPs from the medium.

2. The method according to claim 1, wherein the NRP is selected from the group consisting of vancomycin, cyclosporine A, echinomycin, and triostin A.

3. The method according to claim 1, wherein the NRP is vancomycin.

4. The method according to claim 1, wherein the NRP is cyclosporine A.

5. The method according to claim 1, wherein the NRP is echinomycin.

6. The method according to claim 1, wherein the NRP is triostin A.

7. The method according to claim 1, wherein said bacterium is cultured in medium comprising quinoxaline.

8. The method according to claim 1, wherein said bacterium is cultured in medium comprising quinoxaline-2-carboxylic acid.

9. A method of producing biologically active forms of nonribosomal peptides (NRPs) in Escherichia coli (E. coli) comprising (a) culturing an E. coli bacterium which has an ability to produce NRPs in a medium, and (b) collecting the target NRPs from the medium, wherein said bacterium is modified to increase the production of NRPs as compared to an unmodified bacterium.

10. The method according to claim 9, wherein the NRP is selected from the group consisting of vancomycin, cyclosporine A, echinomycin, and triostin A.

11. The method according to claim 9, wherein the NRP is vancomycin.

12. The method according to claim 9, wherein the NRP is cyclosporine A.

13. The method according to claim 9, wherein the NRP is echinomycin.

14. The method according to claim 9, wherein the NRP is triostin A.

15. The method according to claim 9, wherein said bacterium is cultured in medium comprising quinoxaline.

16. The method according to claim 9, wherein said bacterium is cultured in medium comprising quinoxaline-2-carboxylic acid.

17. An E. coli bacterium capable of producing biologically active forms of nonribosomal peptides (NRPs) comprising multi-plasmid assembly and multi-monocistronic gene assembly.

18. An E. coli bacterium according to claim 17, wherein said multi-monocistronic gene assembly is selected from at least 2 or more of the group of genes comprising ecm1, ecm2, ecm 3, ecm4, ecm 6; ecm7, ecm8, ecm11, ecm12, ecm13, ecm 14, ecm 16, ecm17, ecm18, and fab.

19. An E. coli bacterium according to claim 17, wherein said multi-plasmid assembly is selected from at least 2 or more of the group of plasmids comprising pKW532, pKW538, pKW539, and pKW541.
